(19) United States
(12) Patent Application Publication
(10) Pub. No.: US 2026/0034027 A1
(43) Pub. Date: Feb. 5, 2026

(54) Containers Having Biocompatible Interior Surfaces and Blood Processing Systems Including the Same

(71) Applicant: Terumo BCT, Inc., Lakewood, CO (US)

(72) Inventors: Tyler WINGFIELD, Centennial, CO (US); Bruce GIBBS, Arvada, CO (US); Katherine COLAGROSSO, Lakewood, CO (US); Sayudh GHOSH, Lakewood, CO (US)

(21) Appl. No.: 18/788,618

(22) Filed: Jul. 30, 2024

(51) Int. Cl. A61J 1/14; A61J 1/10; A61M 1/02; B04B 5/04

(52) U.S. Cl. CPC A61J 1/1468; A61J 1/10; A61M 1/0272; B04B 5/0428

(57) ABSTRACT

A container, e.g., a bag, for processing whole blood or a whole blood component and a system for processing whole blood or a whole blood component including the container. The container includes a body and an interior chamber. The body has a bulk portion and a surface portion defining an interior surface of the body. The bulk portion of the body is formed from a polymeric composition comprising a thermoplastic polymer. The surface portion of the body includes a biocompatible material that reduces platelet activation, platelet aggregation, or adhesion of platelets to the interior surface of the body. The interior chamber is at least partially defined by the interior surface of the body.

### FIELD

The present disclosure relates to containers with biocompatible interior surfaces for blood collection and blood processing systems.

### BACKGROUND

This section provides background information related to the present disclosure which is not necessarily prior art.

Whole blood from a donor is typically separated into three components: plasma, red blood cells, and platelets. Whole blood is usually collected from a donor in a bag made of a flexible polymer. A centrifugation process is oftentimes used to separate the whole blood components from one another due to their differing densities. The centrifugation process may occur in the same vial or bag in which the blood was collected and may be performed at a rotational speed between 1,900 revolutions per minute (rpm) and 6,500 rpm such that the blood experiences a relative centrifugal force of between 400 to 2000 times the force of gravity (g). After the centrifugation process, depending on the type of separation protocol employed, the whole blood components may be arranged in three distinct layers, with the relatively dense red blood cells collecting in a layer at a bottom or outermost radial portion of the bag, the less dense plasma collecting in a layer at a top or innermost radial portion of the bag, and a buffy coat layer extending between the top and bottom layers primarily comprising white blood cells and platelets. The white blood cells and platelets may be further separated from each other by transferring the buffy coat layer to another vessel and then performing an additional centrifugation step.

In some centrifugation processes, the bag is positioned at an acute angle with respect to the axis of rotation of the centrifuge such that the heavier whole blood components (e.g., the red blood cells and platelets) accelerate toward and accumulate against a back wall of the bag before sliding against the wall toward the bottom of the bag. In other centrifugation processes, the bag is oriented in a generally horizontal position perpendicular to the axis of rotation of the centrifuge, which allows the whole blood components to be separated into layers with fewer of the components contacting the walls of the bag during the process. After the centrifugation process, the various whole blood components may be separated from one another by sequentially expressing each of the layers from the bag. As the layers are expressed from the bag during the separation process or during subsequent processing steps, the whole blood components may contact and slide against the walls of the bag.

Whole blood collection and separation processes may affect the integrity and/or functionality of certain components of whole blood. For example, such processes may result in aggregation, adhesion, fragmentation, and/or activation of platelets, which may compromise their viability and functionality. Exposure to artificial surfaces, such as the interior surfaces of bags or other containers used in the whole blood separation processes, has been identified as one proposed trigger for platelet activation. Therefore, to improve the recovery of viable platelets, as well as other whole blood components, from whole blood separation processes, it may be desirable to develop bags and other containers for the collection, processing, transfer, and/or storage of whole blood components that have more biocompatible and/or innocuous interior surfaces.

### SUMMARY

This section provides a general summary of the disclosure and is not a comprehensive disclosure of its full scope or all of its features.

A container for processing whole blood or a whole blood component comprises a body and an interior chamber. The body has a bulk portion and a surface portion defining an interior surface of the body. The bulk portion of the body is formed from a polymeric composition comprising a thermoplastic polymer. The surface portion comprises a biocompatible material that reduces platelet activation, platelet aggregation, or adhesion of platelets to the interior surface of the body. The interior chamber is at least partially defined by the interior surface of the body.

The thermoplastic polymer may comprise polyvinyl chloride, polymethyl methacrylate, acrylonitrile butadiene styrene, polyethylene terephthalate, polycarbonate, ethylene-vinyl acetate, a fluoropolymer, a thermoplastic polyolefin, or a combination thereof.

The thermoplastic polymer may comprise polyvinyl chloride. In such case, the polymeric composition may further comprise a plasticizer. The plasticizer may constitute, by weight, greater than or equal to about 20% and less than or equal to about 50% of the polymeric composition.

The plasticizer may comprise one or more of di-(2-ethylhexyl)-phthalate (DEHP), tri-(2-ethyhexyl)-trimellitate (TEHTM), butyryl-tri-n-hexyl-citrate (BTHC), acetyl-tri-n-butyryl-citrate (ATBC), diisononyl cyclohexane-1,2-dicarboxylate (DINCH), and di-(2-ethylhexyl)-adipate (DEHA).

The polymeric composition may be prepared by a dry blending, wet blending, melt blending, compounding, or melt extrusion process.

In aspects, the polymeric composition may further comprise the biocompatible material. In such case, the biocompatible material may comprise a polyester modified siloxane.

The biocompatible material may constitute, by weight, greater than or equal to about 0.5% and less than or equal to about 10% of the polymeric composition.

The surface portion may define the interior surface and an opposite exterior surface of the body and, in such case, the bulk portion may be disposed between the surface portion defining the interior surface of the body and the surface portion defining the exterior surface of the body. The bulk portion and the surface portion of the body may be of unitary one-piece construction and, during or after formation of the body, the biocompatible material may migrate toward the interior surface and the exterior surface of the body such that the surface portion of the body has a relatively high concentration of the biocompatible material, as compared to the concentration of the biocompatible material in the bulk portion of the body.

The bulk portion and the surface portion of the body may be formed by extruding the polymeric composition into a desired shape.

In aspects, the surface portion defining the interior surface of the body may be a coating disposed on the bulk portion of the body. In such case, the coating may comprise the biocompatible material and the biocompatible material may comprise poly (2-methoxyethyl acrylate).

The coating may have a thickness of greater than or equal to about 10 micrometers and less than or equal to about 2 millimeters.

The container may further comprise an anticoagulant disposed in the interior chamber and in physical contact with the interior surface of the body.

In embodiments, the container may be a bag for processing whole blood into two or more whole blood components. In such case, the body may be defined by a first polymeric sheet and a second polymeric sheet joined together by a weld joint.

A system for processing whole blood or a whole blood component comprises a centrifuge, a processing bag positioned in the centrifuge and comprising a body and an interior chamber, and whole blood or a whole blood component comprising platelets contained in the interior chamber of the processing bag. The centrifuge has an axis of rotation and is configured to rotate. The body of the processing bag having a bulk portion and a surface portion defining an interior surface of the body. The interior chamber of the processing bag being at least partially defined by the interior surface of the body. The bulk portion of the body being formed from a polymeric composition comprising a thermoplastic polymer. The whole blood or the whole blood component contained in the interior chamber of the processing bag is in physical contact with the interior surface of the body. The surface portion of the body comprises a biocompatible material that reduces activation, aggregation, or adhesion of the platelets to the interior surface of the body when the centrifuge is rotating and the platelets are being forced in contact with the interior surface of the body.

The thermoplastic polymer may comprise polyvinyl chloride, polymethyl methacrylate, acrylonitrile butadiene styrene, polyethylene terephthalate, polycarbonate, ethylene-vinyl acetate, a fluoropolymer, a thermoplastic polyolefin, or a combination thereof.

The biocompatible material may comprise a polyester modified siloxane, and the polymeric composition may further comprise the biocompatible material. The surface portion of the body may define the interior surface and an opposite exterior surface of the body and, in such case, the bulk portion may be disposed between the surface portion defining the interior surface of the body and the surface portion defining the exterior surface of the body. The bulk portion and the surface portion of the body may be of unitary one-piece construction and, during or after formation of the body, the biocompatible material may migrate toward the interior surface and the exterior surface of the body such that the surface portion of the body has a relatively high concentration of the biocompatible material, as compared to the concentration of the biocompatible material in the bulk portion of the body.

In aspects, the biocompatible material may comprise poly (2-methoxyethyl acrylate), the surface portion defining the interior surface of the body may be a coating disposed on the bulk portion of the body, and the coating may comprise the biocompatible material.

The system may further comprise a platelet bag configured to receive platelets expressed from the processing bag while the centrifuge is rotating. The platelet bag may comprise a platelet bag body and an interior chamber. The platelet bag body may have a bulk portion and a surface portion defining an interior surface of the platelet bag body. The interior chamber of the platelet bag may be at least partially defined by the interior surface of the platelet bag body. Platelets expressed from the processing bag may be received in the interior chamber of the platelet bag. The bulk portion of the platelet bag body may be formed from a polymeric composition comprising a thermoplastic polymer comprising polyvinyl chloride, polymethyl methacrylate, acrylonitrile butadiene styrene, polyethylene terephthalate, polycarbonate, ethylene-vinyl acetate, a fluoropolymer, a thermoplastic polyolefin, or a combination thereof. The surface portion of the platelet bag body may comprise a biocompatible material that reduces activation, aggregation, or adhesion of the platelets to the interior surface of the platelet bag body.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations and are not intended to limit the scope of the present disclosure.

FIG. 1 is a schematic depiction of a container for containing whole blood or a whole blood component.

FIG. 2 is schematic cross-section view of the container of FIG. 1 taken along line 2-2.

FIG. 3 is schematic enlarged view of a portion of the container of FIG. 2 taken generally from rectangle 3 of FIG. 2.

FIG. 4 is a schematic depiction of a system for processing whole blood or a whole blood component comprising a centrifuge and a whole blood processing bag positioned in the centrifuge.

FIG. 5 is a schematic cross-section view of the whole blood processing bag of FIG. 4.

Corresponding reference numerals indicate corresponding parts throughout the several views of the drawings.

### DETAILED DESCRIPTION

Example embodiments will now be described more fully with reference to the accompanying drawings.

The containers described herein have biocompatible or innocuous interior surfaces that can reduce platelet activation, platelet aggregation, and/or adhesion of platelets thereto. The containers may be used in blood processing systems in which at least one whole blood component (e.g., platelets) contained in the containers is subjected to a high centrifugal force that directs the at least one whole blood component toward and in physical contact with the interior surface of the container. In embodiments, the containers may be bags or tubing used for collecting, processing, transferring, and/or storing whole blood or a whole blood component.

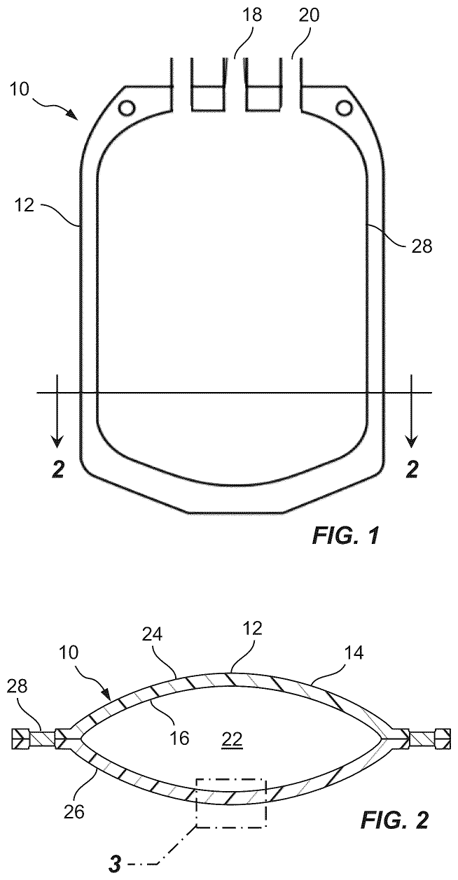

FIGS. 1, 2, and 3 depict a container 10 for containing whole blood or a whole blood component, e.g., platelets, red blood cells, plasma, or residual leukocytes. The container 10 may be used for the collection, processing, transfer, and/or storage of whole blood or a whole blood component. For example, the container 10 may be used for processing whole blook into two or more whole blood components. As shown in FIG. 1, in some embodiments, the container 10 may be a whole blood processing bag in which whole blood is collected from a donor and processed into its components (i.e., via centrifugation) and/or the container 10 may be a platelet bag that receives platelets expressed from a whole blood processing bag during a whole blood separation process. In embodiments, the container 10 may be part of a blood bag set configured for use during processing of whole blood into two or more components using the REVEOS® Automated Blood Processing System manufactured by Terumo BCT of Lakewood, Colorado, USA. In some embodiments, the container 10 may be a vial, tube, or other device used in the collection, processing, transfer, and/or storage of whole blood or whole blood components.

The container 10 comprises a body 12 having an exterior surface 14, an interior surface 16, an inlet port 18, and at least one outlet port 20. The interior surface 16 of the body 12 at least partially defines an interior chamber 22 in which whole blood or a component thereof is received. The interior chamber 22 is surrounded by the body 12 and may be entirely enclosed within the body 12. Whole blood received in the interior chamber 22 is in direct physical contact with the interior surface 16 of the body 12.

In aspects, the container 10 may further comprise an anticoagulant (not shown) disposed in the interior chamber 22 and in direct physical contact with the interior surface 16 of the body 12. The anticoagulant may comprise a citrate-based solution, e.g., citrate phosphate dextrose (CPD), citrate phosphate dextrose adenine (CPDA-1), or a combination thereof. The citrate-based solution may comprise a solution of sodium citrate dihydrate, citric acid monohydrate, monobasic sodium phosphate dihydrate, dextrose monohydrate, adenine, or a combination thereof.

In embodiments, the container 10 may be sterilized. For example, the interior surface 16 of the body 12 of the container 10 may be steam sterilized at a temperature of greater than or equal to about 120 degrees Celsius (° C.), or optionally greater than or equal to about 121° C. and less than or equal to about 140° C., or optionally less than or equal to about 132° C. for a duration of greater than or equal to about 15 minutes and less than or equal to about 30 minutes at a pressure of about 1 Atmosphere (Atm).

The body 12 of the container 10 may be made of a translucent, flexible material. As shown in FIG. 2, the body 12 of the container 10 may be formed by from a first polymeric sheet 24 and a second polymeric sheet 26 that are joined together along their peripheries by a solid weld joint 28. The first polymeric sheet 24 may define a first side of the container 10 and may define a portion of the exterior surface 14 and a portion of the interior surface 16 of the body 12. The second polymeric sheet 26 may define an opposite second side of the container 10 and may define a remaining portion of the exterior surface 14 and a remaining portion of the interior surface 16 of the body 12. The first polymeric sheet 24 and the second polymeric sheet 26 each may have a thickness of greater than or equal to about 0.3 millimeter (mm) and less than or equal to about 5 mm. In aspects, the first polymeric sheet 24 and the second polymeric sheet 26 each may have a thickness of about 0.35 mm. The first polymeric sheet 24 and the second polymeric sheet 26 may be fused or welded together using a radio frequency (RF) welding process. The interior chamber 22 is defined by and between the first polymeric sheet 24 and the second polymeric sheet 26. The weld joint 28 may have at least one dimension greater than or equal to about 1 mm, optionally greater than or equal to about 2 mm, optionally greater than or equal to about 3 mm, optionally greater than or equal to about 4 mm, or optionally greater than or equal to about 5 mm and less than or equal to about 10 mm, optionally less than or equal to about 9 mm, optionally less than or equal to about 8 mm, or optionally less than or equal to about 7 mm.

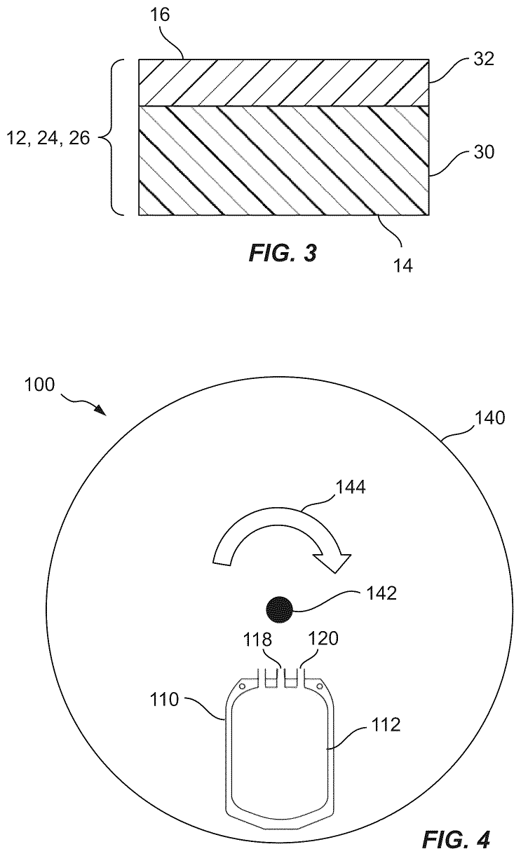

As shown in FIG. 3, the body 12 of the container 10 has a bulk portion 30 and a surface portion 32 that defines the interior surface 16 of the body 12. In aspects, the first polymeric sheet 24 and the second polymeric sheet 26 each may have a bulk portion 30 and a surface portion 32 that defines a portion of the interior surface 16 and a portion of the exterior surface 14 of the body 12, with the polymeric sheet 24 and the second polymeric sheet 26 together defining the entire interior surface 16 and the entire exterior surface 14 the body 12.

The bulk portion 30 of the body 12 is configured to provide the body 12 with mechanical stability and the ability to form a fluid-tight seal around the interior chamber 22. The bulk portion 30 (and optionally the surface portion 32) of the body 12 may be formed from and may comprise a polymeric composition that comprises a thermoplastic polymer, a plasticizer, and optionally a biocompatible material. In addition, the polymeric composition optionally may comprise an additive, e.g., a lubricant, heat stabilizer, light stabilizer, pigment, filler, density modifier, or a combination thereof.

The thermoplastic polymer may comprise any polymer that can be formed into a container, e.g., a bag. For example, the thermoplastic polymer may comprise polyvinyl chloride (PVC), polymethyl methacrylate (PMMA), acrylonitrile butadiene styrene (ABS), polyethylene terephthalate (PET), polycarbonate (PC), ethylene-vinyl acetate (EVA), a fluoropolymer, a thermoplastic polyolefin (PO) (also referred to as a polyalkene), or a combination thereof. Examples of polyolefins include polypropylene (PP), polyethylene (e.g., high density polyethylene, HDPE), polybutene-1 (PB-1), polymethylpentene (PMP), ethylene-octene copolymers, stereo-block PP, olefin block copolymers, propylene-butane copolymers, cyclic olefin copolymers (COC), cyclic olefin polymers (COC), and combinations thereof. In aspects, the thermoplastic polymer may comprise polyvinyl chloride (PVC). The thermoplastic polymer may constitute, by weight, greater than or equal to about 40%, optionally greater than or equal to about 50%, optionally greater than or equal to about 55%, or optionally greater than or equal to about 60% and less than or equal to about 80%, optionally less than or equal to about 70%, or optionally less than or equal to about 65% of the polymeric composition.

The plasticizer may include a cyclohexane-based material. For example, in aspects, the plasticizer may include one or more of di-(2-ethylhexyl)-phthalate (DEHP), tri-(2-ethyhexyl)-trimellitate (TEHTM), butyryl-tri-n-hexyl-citrate (BTHC), a dialkyl cyclohexane-1,2-dicarboxylate such as diisononyl cyclohexane-1,2-dicarboxylate (DINCH), and di-(2-ethylhexyl)-adipate (DEHA). Alternatively, the plasticizer may comprise a non-cyclohexane-based plasticizer such as acetyl-tri-n-butyryl-citrate (ATBC). For example, in aspects, the plasticizer may comprise 1,2-cyclohexane dicarboxylic acid diisononyl ester, also known as diisononyl cyclohexane-1,2-dicarboxylate (DINCH) (CAS No. 166412-78-8). In aspects, the bulk portion 30 of the body 12 may be substantially free of phthalates, terephthalates, and combinations thereof. For example, the bulk portion 30 of the body 12 may be substantially free of bis(2-ethylhexyl) terephthalate (DEHT) (CAS No. 6422-86-2). The plasticizer may constitute, by weight, greater than or equal to about 20%, optionally greater than or equal to about 25%, optionally greater than or equal to about 30%, or optionally greater than or equal to about 35% and less than or equal to about 50%, optionally less than or equal to about 45%, or optionally less than or equal to about 40% of the polymeric composition. The plasticizer has high resistance to leaching. As such, neither the plasticizer nor its derivatives will volatilize or be extracted from the polymeric composition or from the body 12 of the container 10 in appreciable amounts and thus will not contaminate whole blood, whole blood components, or anticoagulants disposed in the interior chamber 22 of the container 10, even after prolonged exposure thereto.

The polymeric composition may be prepared using a dry blending, wet blending, melt blending, compounding, melt extrusion process, or a combination thereof. Such processes may be performed at a temperature of greater than or equal to about 100 degrees Celsius and less than or equal to about 300 degrees Celsius. The specific process used to prepare the polymeric composition may depend on the composition of the thermoplastic polymer and/or the plasticizer.

The bulk portion 30 (and optionally the surface portion 32) of the body 12 may be formed from the polymeric composition via any suitable method. For example, in aspects, the bulk portion 30 (and optionally the surface portion 32) of the body 12 may be formed from the polymeric composition via an extrusion process. For example, the bulk portion 30 (and optionally the surface portion 32) of the body 12 may be formed by extruding the polymeric composition into a desired shape, e.g., into the form of a sheet or tube. In aspects, the bulk portion 30 (and optionally the surface portion 32) of the body 12 may be formed from the polymeric composition via a blown film extrusion process, wherein the polymeric composition is extruded into the shape of a relatively thick tube and then air is blown into the tube to produce a relatively thin film.

In aspects, the polymeric composition may be formulated to provide the body 12 of the container 10 with the ability to maintain its structural integrity during heat sterilization processes. Additionally or alternatively, the polymeric composition may be formulated to provide the body 12 of the container 10 with the ability to allow the first polymeric sheet 24 and the second polymeric sheet 26 to be joined together by being melted, fused, or welded together by heating. As such, in aspects, the polymeric composition may have a melting point of greater than or equal to about 130° C., optionally greater than or equal to about 140° C., or optionally greater than or equal to about 150° C. and less than or equal to about 300° C. The polymeric composition may be formulated to provide the body 12 of the container 10 with the ability to maintain its structural integrity without leaking when the body 12 of the container 10 is filled with liquid (e.g., 530 milliliters), formed with a 3-millimeter weld along its periphery, and subjected to flash freezing conditions (e.g., at temperatures of about −50° C.), extending freezing temperatures (e.g., temperatures of about −80° C. for greater than or equal to about 24 hours), freezing followed by rapid thawing (e.g., at temperatures of about 37° C.), centrifugation (e.g., at a centrifugal force of about 5,018 g for a duration of about 10 minutes), compression (at about 7.25 pounds per square inch), and dropping from a height of about 5 feet.

The surface portion 32 of the body 12 comprises a biocompatible material that is configured to reduce platelet activation, platelet aggregation, and/or adhesion of platelets to the interior surface 16 of the body 12. As such, inclusion of the biocompatible material in the surface portion 32 and on the interior surface 16 of the body 12 can allow for more effective and higher quality processing of whole blood and may result in a higher yield of platelets from the whole blood separation process. For example, reducing platelet activation, platelet aggregation, and/or adhesion of platelets to the interior surface 16 of the body 12 can help prevent loss of platelets during the whole blood separation process, resulting in a higher yield of platelets. Preventing platelets from adhering or sticking to the interior surface 16 of the body 12 may help prevent residual platelets from remaining in the container 10 during the whole blood separation process and undesirably mixing with the other whole blood components.

The biocompatible material included in the surface portion 32 of the body 12 is formulated to provide the interior surface 16 of the body 12 with biocompatibility and innocuous surface properties to improve the collection, processing, and storage of whole blood and whole blood components without substantially altering the mechanical and thermal properties of the body 12, specifically, of the polymeric composition used to form the bulk portion 30 (and optionally the surface portion 32) of the body 12. The specific biocompatible material included in the surface portion 32 of the body 12 may depend on the manner in which the surface portion 32 of the body 12 is formed. In aspects, the surface portion 32 and the bulk portion 30 of the body 12 may be of unitary one-piece construction and may be formed substantially simultaneously from the same polymeric composition and, in such case, the surface portion 32 of the body 12 will comprise a first biocompatible material. In aspects, the surface portion 32 and the bulk portion 30 may be discrete components of the body 12, with the surface portion 32 being formed in a discrete step after formation of the bulk portion 30 of the body 12 and, in such case, the surface portion 32 of the body 12 will comprise a second biocompatible material. The composition of the first biocompatible material is different that than of the second biocompatible material.

In embodiments where the bulk portion 30 and the surface portion 32 of the body 12 are of unitary one-piece construction and are formed from the same polymeric composition, in addition to the thermoplastic polymer, plasticizer, and the optional additive, the polymeric composition further comprises the first biocompatible material. The first biocompatible material may be formulated such that the interior surface 16 of the body 12 is hydrophobic. The first biocompatible material may comprise a polyester modified siloxane. For example, the first biocompatible material may comprise a copolymer of polydimethylsiloxane ([—Si(CH₃)₂)O—]ₙ) and an aliphatic polyester ([—R—COO—]ₘ), where n and m is the number of repeating monomer [—Si(CH₃)₂)O—]ₙ or ([—R—COO—]ₘ units. Specific examples of a polyester modified siloxane are TEGOMER® manufactured by Evonik Corporation and SILIMER™ manufactured by Silike Tech. The first biocompatible material may constitute, by weight, greater than or equal to about 0.5%, optionally greater than or equal to about 1%, or optionally greater than or equal to about 1.5% and less than or equal to about 10%, optionally less than or equal to about 8%, optionally less than or equal to about 5%, optionally less than or equal to about 3%, or optionally less than or equal to about 2% of the polymeric composition. The first biocompatible material has high resistance to leaching. As such, neither the first biocompatible material nor its derivatives will volatilize or be extracted from the polymeric composition or from the body 12 of the container 10 in appreciable amounts and thus will not contaminate whole blood, whole blood components, or anticoagulants disposed in the interior chamber 22 of the container 10, even after prolonged exposure thereto.

In embodiments where the bulk portion 30 and the surface portion 32 of the body 12 are of unitary one-piece construction and are formed from the same polymeric composition, the bulk portion 30 and the surface portion 32 of the body 12 may be formed by extruding the polymeric composition comprising the first biocompatible material into a desired shape. For example, the polymeric composition comprising the first biocompatible material may be extruded into the shape of a sheet (e.g., into the shape of the first polymeric sheet 24 and/or the second polymeric sheet 26). And, during or after the polymeric composition is extruded into a desired shape, a portion of the first biocompatible material included in the polymeric composition will migrate toward and to the interior surface 16 of the body 12 such that the surface portion 32 of the body 12 has a relatively high concentration of the first biocompatible material, as compared to the concentration of the first biocompatible material in the bulk portion 30 of the body 12. For example, after the bulk portion 30 and the surface portion 32 of the body 12 are extruded into a desired shape, a concentration of the first biocompatible material in the surface portion 32 of the body 12 extending along the interior surface 16 thereof may be at least two times greater than, or optionally at least three times greater than the concentration of the first biocompatible material in the bulk portion 30 of the body 12. During or after the polymeric composition is extruded into a desired shape, a portion of the first biocompatible material included in the polymeric composition may also migrate toward the exterior surface 14 of the body 12. In such case, the surface portion 32 of the body 12 may define both the interior surface 16 and the exterior surface 14 of the body 12, with the bulk portion 30 of the body 12 extending therebetween.

In embodiments where the surface portion 32 and the bulk portion 30 are discrete components of the body 12, the surface portion 32 of the body 12 is in the form of a coating comprising the second biocompatible material and deposited on the bulk portion 30 of the body 12 after formation thereof. The second biocompatible material may comprise a chemical compound having a hydrophilic moiety and a hydrophobic moiety, with the hydrophobic moiety being formulated to help the second biocompatible material adhere to the bulk portion 30 of the body 12 and the hydrophilic moiety being formulated such that the interior surface 16 of the body 12 is hydrophilic. In aspects, the second biocompatible material may comprise poly (2-methoxyethyl acrylate) (PMEA) (CAS No. 28628-64-0). In aspects where the surface portion 32 of the body 12 is in the form of a coating comprising the second biocompatible material, the coating may have a thickness of greater than or equal to about 10 micrometers and less than or equal to about 2 millimeters.

The coating comprising the second biocompatible material may be formed on the bulk portion 30 of the body 12 by depositing a precursor solution comprising the second biocompatible material and a solvent on the bulk portion 30 of the body 12 and then removing the solvent. In aspects, the solvent may comprise a polar solvent, e.g., methanol. The second biocompatible material may constitute, by weight, greater than or equal to about 0.1%, optionally greater than or equal to about 0.5%, optionally greater than or equal to about 1%, or optionally greater than or equal to about 1.5% and less than or equal to about 10%, optionally less than or equal to about 8, optionally less than or equal to about 5%, optionally less than or equal to about 3%, or optionally less than or equal to about 2% of the precursor solution.

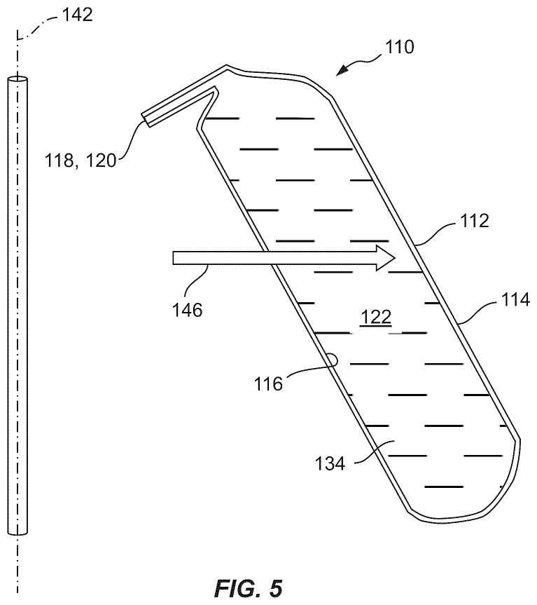

FIGS. 4 and 5 depict a system 100 for processing whole blood comprising a centrifuge 140 and a container in the form of a whole blood processing bag 110 positioned in the centrifuge 140. The bag 110 depicted in FIGS. 4 and 5 is similar in many respects to the container 10 depicted in FIGS. 1, 2, and 3 and like numerals between the figures generally designate like or corresponding elements; description of common subject matter between the container 10 and the bag 110 generally may not be repeated here. In embodiments, the bag 110 positioned in the centrifuge 140 may be a platelet bag, a red blood cell bag, plasma bag, residual leukocyte bag, or sampling bag. In other embodiments, the bag 110 may be a vial, tube, or other container positioned within a centrifuge 140 and subjected to a centrifugal force during a blood separation process.

The centrifuge 140 has an axis of rotation 142 and is configured to rotate (e.g., in the direction of arrow 144) during operation of the system 100 to facilitate processing or separation of whole blood contained in the bag 110 into its components (e.g., platelets, red blood cells, plasma, and residual leukocytes) due to the differing densities of the components. For example, the centrifuge 140 may be configured to rotate at a rotational speed of greater than or equal to about 1,900 revolutions per minute (rpm), optionally greater than or equal to about 2,000 rpm, optionally greater than or equal to about 2,500 rpm, or optionally greater than or equal to about 3,000 rpm and less than or equal to about 6,000 rpm, optionally less than or equal to about 5,000 rpm, or optionally less than or equal to about 4,000 rpm.

The processing bag 110 comprises a body 112 having an exterior surface 114, an interior surface 116, an inlet port 118, and at least one outlet port 120. The interior surface 116 of the body 112 at least partially defines an interior chamber 122 in which whole blood 134 or a component thereof is received and contained. The interior chamber 122 is surrounded by the body 112 and may be entirely enclosed within the body 112. Whole blood 134 disposed in the interior chamber 122 is in direct physical contact with the interior surface 116 of the body 112. Like the body 12, the body 112 of the bag 110 likewise may have a bulk portion (not shown) and a surface portion (not shown) that defines the interior surface 116 of the body 112. The bulk portion and the surface portion of the body 112 may be made of substantially the same materials using substantially the same methods described above with respect to the bulk portion 30 and the surface portion 32 of the body 12. Like the surface portion 32 of the body 12, the surface portion of the body 112 comprises a biocompatible material that reduces activation, aggregation, or adhesion of the platelets to the interior surface 116 of the body 112. Like the container 10, the bag 110 may be sterilized prior to receiving whole blood 134 or a whole blood component in the interior chamber 122 and may further comprise an anticoagulant (not shown) disposed in the interior chamber 122 and in direct physical contact with the interior surface 116 of the body 112.

During operation of the system 100 and rotation of the centrifuge 140, components of the whole blood 134 disposed in the interior chamber 122 of the bag 110 experience a centrifugal force in the direction of arrow 146 that forces the components (e.g., the platelets) in contact with the interior surface 116 of the body 112. For example, the components of the whole blood 134 disposed in the interior chamber 122 of the bag 110 may experience a relative centrifugal force of greater than or equal to about 600 times the force of gravity (g), optionally greater than or equal to about 700 g, optionally greater than or equal to about 1,000 g, optionally greater than or equal to about 1,500 g, optionally greater than or equal to about 2,000 g, or optionally greater than or equal to about 3,000 g and less than or equal to about 6,000 g, optionally less than or equal to about 5,000 g, optionally less than or equal to about 4,500 g, optionally less than or equal to about 4,000 g, or optionally less than or equal to about 3,500 g.

The biocompatible material included in the surface portion of the body 112 reduces undesirable interaction between whole blood components (e.g., the platelets) disposed in the interior chamber 122 of the bag 110 and the interior surface 116 of the body 112, which may help prevent activation, aggregation, or adhesion of the platelets to the interior surface 116 of the body 112. Use of the container 10, 110 in applications where whole blood 134 or a whole blood component disposed in the interior chamber 122 of the bag 110 is subjected to a centrifugal force, e.g., during processing, may be particularly beneficial as the biocompatible material included in the surface portion of the body 112 may help reduce undesirable interaction between the whole blood 134 or the whole blood component and the interior surface 116 of the body 112.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

The terminology used herein is for the purpose of describing example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended terms “comprises,” “comprising,” “including,” and “having,” are to be understood as non-restrictive terms used to describe and claim various embodiments set forth herein, in certain aspects, the terms may alternatively be understood to instead be a more limiting and restrictive term, such as “consisting of” or “consisting essentially of.” Thus, for any given embodiment reciting compositions, materials, components, elements, ingredients, features, integers, operations, and/or process steps, the present disclosure also specifically includes embodiments consisting of, or consisting essentially of, such recited compositions, materials, components, elements, ingredients, features, integers, operations, and/or process steps. In the case of “consisting of,” the alternative embodiment excludes any additional compositions, materials, components, elements, ingredients, features, integers, operations, and/or process steps, while in the case of “consisting essentially of,” any additional compositions, materials, components, elements, ingredients, features, integers, operations, and/or process steps that materially affect the basic and novel characteristics are excluded from such an embodiment, but any compositions, materials, components, elements, ingredients, features, integers, operations, and/or process steps that do not materially affect the basic and novel characteristics can be included in the embodiment.

Any method steps, processes, and operations described herein are not to be construed as necessarily requiring their performance in the order discussed or illustrated, unless specifically identified as an order of performance. It is also to be understood that additional or alternative steps may be employed, unless otherwise indicated.

When a component, element, or layer is referred to as being “on,” “engaged to,” “connected to,” or “coupled to” another element or layer, it may be directly on, engaged, connected or coupled to the other component, element, or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes combinations of one or more of the associated listed items.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer, or section. Terms such as “first,” “second,” and other numerical terms when used herein do not imply a sequence or order unless clearly indicated by the context. Thus, a first step, element, component, region, layer, or section discussed below could be termed a second step, element, component, region, layer, or section without departing from the teachings of the example embodiments.

Spatially or temporally relative terms, such as “before,” “after,” “inner,” “outer,” “beneath,” “below,” “lower,” “above,” “upper,” and the like, may be used herein for ease of description to describe one element or feature's relationship to another element(s) or feature(s), as illustrated in the figures. Spatially or temporally relative terms may be intended to encompass different orientations of the device or system in use or operation in addition to the orientation depicted in the figures.

Throughout this disclosure, the numerical values represent approximate measures or limits to ranges and encompass minor deviations from the given values and embodiments, having about the value mentioned as well as those having exactly the value mentioned. Other than the working examples provided at the end of the detailed description, all numerical values of parameters (e.g., of quantities or conditions) in this specification are to be understood as being modified in all instances by the term “about” whether or not “about” actually appears before the numerical value. Numerical values of parameters in the appended claims are to be understood as being modified by the term “about” only when such term appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%. In addition, disclosure of ranges includes disclosure of all values and further divided ranges within the entire range, including endpoints and sub-ranges given for the ranges.

As used herein, the terms “composition” and “material” are used interchangeably to refer broadly to a substance containing at least the preferred chemical constituents, elements, or compounds, but which may also comprise additional elements, compounds, or substances, including trace amounts of impurities, unless otherwise indicated. An “X-based” composition or material broadly refers to compositions or materials in which “X” is the single largest constituent of the composition or material on a weight percentage (%) basis. This may include compositions or materials having, by weight, greater than 50% X, as well as those having, by weight, less than 50% X, so long as X is the single largest constituent of the composition or material based upon its overall weight. When a composition or material is referred to as being “substantially free” of a substance, the composition or material may comprise, by weight, less than 5%, optionally less than 3%, optionally less than 1%, or optionally less than 0.1% of the substance.

What is claimed is:

1. A container for processing whole blood or a whole blood component, the container comprising:
a body having a bulk portion and a surface portion defining an interior surface of the body, the bulk portion of the body being formed from a polymeric composition comprising a thermoplastic polymer and the surface portion comprising a biocompatible material that reduces platelet activation, platelet aggregation, or adhesion of platelets to the interior surface of the body; and
an interior chamber at least partially defined by the interior surface of the body.

2. The container of claim 1, wherein the thermoplastic polymer comprises polyvinyl chloride, polymethyl methacrylate, acrylonitrile butadiene styrene, polyethylene terephthalate, polycarbonate, ethylene-vinyl acetate, a fluoropolymer, a thermoplastic polyolefin, or a combination thereof.

3. The container of claim 1, wherein the thermoplastic polymer comprises polyvinyl chloride, wherein the polymeric composition further comprises a plasticizer, and wherein the plasticizer constitutes, by weight, greater than or equal to about 20% and less than or equal to about 50% of the polymeric composition.

4. The container of claim 3, wherein the polymeric composition is prepared by a dry blending, wet blending, melt blending, compounding, or melt extrusion process.

5. The container of claim 1, wherein the polymeric composition further comprises the biocompatible material, and wherein the biocompatible material comprises a polyester modified siloxane.

6. The container of claim 5, wherein the biocompatible material constitutes, by weight, greater than or equal to about 0.5% and less than or equal to about 10% of the polymeric composition.

7. The container of claim 5, wherein the surface portion defines the interior surface and an opposite exterior surface of the body, the bulk portion is disposed between the surface portion defining the interior surface of the body and the surface portion defining the exterior surface of the body, the bulk portion and the surface portion of the body are of unitary one-piece construction, and wherein, during or after formation of the body, the biocompatible material migrates toward the interior surface and the exterior surface of the body such that the surface portion of the body has a relatively high concentration of the biocompatible material, as compared to the concentration of the biocompatible material in the bulk portion of the body.

8. The container of claim 7, wherein the bulk portion and the surface portion of the body are formed by extruding the polymeric composition into a desired shape.

9. The container of claim 1, wherein the surface portion defining the interior surface of the body is a coating disposed on the bulk portion of the body, wherein the coating comprises the biocompatible material, and wherein the biocompatible material comprises poly (2-methoxyethyl acrylate).

10. The container of claim 9, wherein the coating has a thickness of greater than or equal to about 10 micrometers and less than or equal to about 2 millimeters.

11. The container of claim 1, further comprising an anticoagulant disposed in the interior chamber and in physical contact with the interior surface of the body.

12. The container of claim 1, wherein the container is a bag for processing whole blood or a whole blood component.

13. The container of claim 12, wherein the body is defined by a first polymeric sheet and a second polymeric sheet joined together by a weld joint.

14. A system for processing whole blood or a whole blood component, the system comprising:
a centrifuge having an axis of rotation and being configured to rotate;
a processing bag positioned in the centrifuge, the processing bag comprising a body and an interior chamber, the body having a bulk portion and a surface portion defining an interior surface of the body, the interior chamber being at least partially defined by the interior surface of the body, the bulk portion of the body being formed from a polymeric composition comprising a thermoplastic polymer; and
whole blood or a whole blood component contained in the interior chamber of the processing bag and in physical contact with the interior surface of the body, the whole blood or the whole blood component comprising platelets,
wherein the surface portion of the body comprises a biocompatible material that reduces activation, aggregation, or adhesion of the platelets to the interior surface of the body when the centrifuge is rotating and the platelets are being forced in contact with the interior surface of the body.

15. The system of claim 14, wherein the thermoplastic polymer comprises polyvinyl chloride, polymethyl methacrylate, acrylonitrile butadiene styrene, polyethylene terephthalate, polycarbonate, ethylene-vinyl acetate, a fluoropolymer, a thermoplastic polyolefin, or a combination thereof.

16. The system of claim 14, wherein the biocompatible material comprises a polyester modified siloxane, the polymeric composition further comprises the biocompatible material, the surface portion defines the interior surface and an opposite exterior surface of the body, the bulk portion is disposed between the surface portion defining the interior surface of the body and the surface portion defining the exterior surface of the body, the bulk portion and the surface portion of the body are of unitary one-piece construction, and wherein, during or after formation of the body, the biocompatible material migrates toward the interior surface and the exterior surface of the body such that the surface portion of the body has a relatively high concentration of the biocompatible material, as compared to the concentration of the biocompatible material in the bulk portion of the body.

17. The system of claim 16, wherein the thermoplastic polymer comprises polyvinyl chloride, the polymeric composition further comprises a plasticizer that constitutes, by weight, greater than or equal to about 20% and less than or equal to about 50% of the polymeric composition.

18. The system of claim 14, wherein the biocompatible material comprises poly (2-methoxyethyl acrylate), the surface portion defining the interior surface of the body is a coating disposed on the bulk portion of the body, and the coating comprises the biocompatible material.
